(19) United States
(12) Patent Application Publication
(10) Pub. No.: US 2009/0023889 A1
(43) Pub. Date: Jan. 22, 2009

(54) PROCESS FOR PRODUCING ETHER POLYMER

(75) Inventors: Kozo Misumi, Osaka-shi (JP); Yutaka Shiomi, Osaka-shi (JP); Shigeru Shoji, Osaka-shi (JP); Yasushi Hamura, Osaka-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: DAISCO CO., LTD., Osaka-shi (JP)

(21) Appl. No.: 11/916,366

(22) PCT Filed: Jun. 1, 2006
(86) PCT No.: PCT/JP2006/310995

§ 371 Date: Jan. 16, 2008

(30) Foreign Application Priority Data
Jun. 3, 2005 (JP) 2005-163406

(51) Int. Cl. C08G 65/34; C07C 33/04

(52) U.S. Cl. 528/400; 568/855

(57) ABSTRACT

An object of the invention is to provide a process for producing an ether (co)polymer in which molecular weight control is easy and post treatment of the reaction liquid after polymerization (solvent recovery, etc.) is easy; a chain transfer agent to be used in the production process; and an ether (co) polymer obtained according to the production process. The process for producing an ether (co) polymer of the invention is characterized in that an ether monomer is polymerized in a solvent, in the presence of a catalyst comprising a condensate of an organotin compound and an alkyl phosphate, and a chain transfer agent comprising, as the major ingredient thereof, an aliphatic polyalcohol of the following general formula (I), thereby producing an ether (co)polymer. Cₓ(OH)_{y}H_{z}. In the formula, X indicates an integer of from 2 to 8; Y indicates an integer of from 2 to (2X+2); Z indicates an integer of 2X+2−Y; and the hydroxyl groups are bonded at any positions.

### TECHNICAL FIELD

The present invention relates to a process for producing an ether (co)polymer (this means an ether homopolymer and an ether-base polynary copolymer, and the same shall apply hereinunder), in which a condensate of an organotin compound and an alkyl phosphate is used as a catalyst, a chain transfer agent to be used in the production process, and an ether (co)polymer produced according to the production process.

### BACKGROUND ART

Ether (co)polymers, in particular epichlorohydrin rubber (this means a homopolymer of epichlorohydrin, a binary copolymer of epichlorohydrin and ethylene oxide, or a ternary copolymer of epichlorohydrin and ethylene oxide and allyl glycidyl ether, and the same shall apply hereinunder) are much used in various fields as oil-resistant rubber, since they have well-balanced properties of heat resistance, oil resistance, cold resistance, gas permeation resistance, etc. In addition, depending on the manner in which they are processed, rubbers having a different molecular weight are desired.

In producing such ether (co) polymers, it is known to add a chain transfer agent such as water, alcohol, aromatic compound or the like for the purpose of molecular weight control.

For example, Patent Reference 1 (JP-A 2000-319383) discloses a process for producing a hydroxyl group-terminated polyether obtained by cation-polymerizing a monomer that contains an epoxy group and an ethylenic unsaturated group through solution polymerization in the presence of a chain transfer agent such as water, alcohol, aromatic compound or the like and using a latent acid generator as an initiator. This process enables molecular weight control, but the post-treatment step therein for removing the chain transfer agent from the solvent recovered from the reaction mixture after the polymerization is extremely complicated and the agent could not be sufficiently removed.

Specifically, in general, polymerization is attained in a solvent for the reason of easiness in polymerization control and the like, and after the reaction, the reaction mixture is separated into the intended polymer and the solvent; and in general, the recovered solvent is processed by distillation or the like and recycled for reuse in reaction; but in the above-mentioned prior art, the chain transfer agent added during the reaction could not be sufficiently removed, and therefore, there is a drawback in that the chain transfer agent may gradually accumulate in the reaction system after every solvent recovery, thereby having some negative influence on the polymerization reaction.

On the other hand, Patent Reference 2 (U.S. Pat. No. 3,773,694) by the present applicant discloses the following: When an organotin-phosphate condensate is used as a polymerization catalyst, then epichlorohydrin monomer polymerization may be effected in slurry in an aliphatic or alicyclic hydrocarbon solvent, and as compared with a solution polymerization process, this is extremely advantageous in an industrial aspect with respect to simplification of polymerization facilities and to efficiency improvement in post treatment such as separation of the intended product from the reaction liquid after polymerization. However, this reference does not describe molecular weight control by a chain transfer agent and a method of effective removal of a chain transfer agent from the reaction liquid.

### DISCLOSURE OF THE INVENTION

### Problems that the Invention is to Solve

An object of the invention is to provide a process for producing an ether (co)polymer in which molecular weight control is easy and post treatment of the reaction liquid after polymerization (solvent recovery, chain transfer agent removal from solvent) is easy; a chain transfer agent to be used in the production process; and an ether (co)polymer obtained according to the production process.

### Means for Solving the Problems

The present inventors have intensively studied for the purpose of solving the above problems, and as a result, have found out a novel process for producing an ether (co)polymer mentioned below, and have completed the invention.

Specifically, the first process for producing an ether (co) polymer of the invention is characterized in that an ether monomer is polymerized in a solvent, in the presence of a catalyst comprising a condensate of an organotin compound and an alkyl phosphate, and a chain transfer agent comprising, as the major ingredient thereof, an aliphatic polyalcohol of the following general formula (I), thereby producing an ether (co) polymer.

Cₓ(OH)_{y}H_{z} (I)

[In the formula, X indicates an integer of from 2 to 8; Y indicates an integer of from 2 to (2X+2); Z indicates an integer of 2X+2−Y; and the hydroxyl groups are bonded at any positions.]

The second process for producing an ether (co)polymer of the invention is characterized in that an ether monomer is polymerized in a solvent immiscible with water, in the presence of a catalyst comprising a condensate of an organotin compound and an alkyl phosphate, and a chain transfer agent comprising, as the major ingredient thereof, an aliphatic polyalcohol of the above general formula (I), then the obtained reaction mixture is separated into an ether (co)polymer and a liquid component, and thereafter the liquid component is purified according to a water extraction method, and the solvent is recovered.

In the above first and second processes for ether (co)polymer production, the ether monomer polymerization is preferably slurry polymerization.

The invention also provides a chain transfer agent comprising, as the major ingredient thereof, an aliphatic polyalcohol of the above general formula (I), which is used in the above first and second processes for ether (co) polymer production; and provides an ether (co)polymer produced according to these processes.

### EFFECT OF THE INVENTION

According to the invention, the molecular weight of the ether (co)polymer, or that is, the Mooney viscosity to be an index for determining the processability of the polymer is easy to control, and in addition, the polymerization rate may be effectively prevented from lowering. In addition, after completion of the polymerization, the chain transfer agent may be readily removed from the recovered solvent. Accordingly, the chain transfer agent does not gradually accumulate in the reaction system after every solvent recovery, and the recovered solvent can be repeatedly reused with no problem.

### BEST MODE FOR CARRYING OUT THE INVENTION

Not specifically defined, the ether monomer for use in the invention may be any monomer of, for example, such that, when the oxirane ring in the monomer is ion-polymerized, then the monomer may undergo successive addition polymerization to give a polymer having an increased molecular weight through ether bonding, and it may be a commercial product or may be prepared according to known technology.

Concretely, the following monomers are preferably used.

Examples of a halogen-containing ether monomer (1) include epichlorohydrin, epibromohydrin, etc. Epichlorohydrin is especially preferred.

Examples of a halogen-free ether monomer (2) include ethylene oxide, propylene oxide, butene oxide, styrene oxide, phenyl glycidyl ether, etc. Ethylene oxide is especially preferred.

Also usable is a combination of halogen-free ether monomers (2); for example, a combination of phenyl glycidyl ether and ethylene oxide, and a combination of phenyl glycidyl ether, ethylene oxide and a copolymerizable crosslink site monomer (3).

The copolymerizable crosslink site monomer (3) may be any ether monomer capable of crosslinking the polyether copolymer of the invention, and includes, for example, epihalohydrins such as epichlorohydrin, epibromohydrin, epiiodohydrin; halogen-containing ether monomers such as p-chlorostyrene oxide, dibromophenyl glycidyl ether, m-chloromethylstyrene oxide, p-chloromethylstyrene oxide, glycidyl chloroacetate, chloromethyl glycidate; ethylenic unsaturated group-containing ether monomers such as allyl glycidyl ether, glycidyl acrylate, glycidyl methacrylate, glycidyl crotonate, 3,4-epoxy-1-butene; diepoxy compounds such as 2,3-epoxypropyl-2′,3′-epoxy-2′-methylpropyl ether, glycidyl methaglycidate, methaglycidyl glycidate, 1,2,3,4-diepoxy-2-methylbutane. Two or more such crosslink site monomers (3) may be used, as combined.

In case where two or more monomers are used, as combined, the ratio by weight of the monomers may be suitably determined in accordance with known technology.

The solvent for use in the invention may be one generally used in solution polymerization, slurry polymerization, etc. In slurry polymerization, the solvent may be suitably selected in correlation with the affinity thereof for the intended product; but for easy solid-liquid separation of the reaction mixture into polymer and solvent, preferably used is an aliphatic or alicyclic hydrocarbon. For water extraction, used is a solvent immiscible with water. Preferred examples of the solvent are butane, pentane, hexane, heptane, octane, nonane, decane, dodecane, cyclohexane, methylcyclohexane, petroleum ether, petroleum benzil, ligroin, liquid paraffin, etc. Especially preferred is a solvent of which the boiling point at normal pressure falls within a favorable range for industrial use (for example, 35 to 100° C.), such as pentane, hexane, heptane.

The amount of the solvent to be used may be so determined that the monomer concentration could be within a range of from 3 to 50% by weight relative to the total amount of the monomer and the solvent.

The catalyst to be used in the production process of the invention is a condensate of an organotin compound and an alkyl phosphate.

The organotin compound is selected from compounds of the following general formulae (i) to (iv):

RₐSnX₄₋ₐ (i)

[In the formula, R represents a group selected from the group consisting of an alkyl group having from 1 to 12 carbon atoms and optionally having a substituent, an alkenyl group having from 2 to 12 carbon atoms, a cycloalkyl group having from 3 to 8 carbon atoms, an aryl group, an aryl group substituted by an alkyl group having from 1 to 4 carbon atoms, and an aralkyl group having 7 or 8 carbon atoms; X represents an atom or a group selected from the group consisting of a halogen atom, an alkoxy group having from 1 to 12 carbon atoms, an aryloxy group, an acyloxy group having from 2 to 18 carbon atoms and its partial ester residue; a indicates an integer of from 1 to 4; and when a is 1, three X's may be the same or different, when a is 2, two R's and two X's may be the same or different, and when a is 3 or 4, plural R s may be the same or different.]

R_{b}SnO_{c} (ii)

[In the formula, R represents a group selected from the group consisting of an alkyl group having from 1 to 12 carbon atoms and optionally having a substituent, an alkenyl group having from 2 to 12 carbon atoms, a cycloalkyl group having from 3 to 8 carbon atoms, an aryl group, an aryl group substituted by an alkyl group having from 1 to 4 carbon atoms, and an aralkyl group having 7 or 8 carbon atoms; b indicates an integer of 1 or 2; and when b is 1, c is 3/2, when b is 2, c is 1.]

R¹(R⁰₂SnOSnR⁰₂)R¹ (iii)

[In the formula, R⁰ represents a group selected from the group consisting of an alkyl group having from 1 to 12 carbon atoms and optionally having a substituent, an alkenyl group having from 2 to 12 carbon atoms, a cycloalkyl group having from 3 to 8 carbon atoms, an aryl group, an aryl group substituted by an alkyl group having from 1 to 4 carbon atoms, and an aralkyl group having 7 or 8 carbon atoms; R¹ represents an atom or a group selected from the group consisting of an alkyl group having from 1 to 12 carbon atoms and optionally having a substituent, an alkenyl group having from 2 to 12 carbon atoms, a cycloalkyl group having from 3 to 8 carbon atoms, an aryl group, an aryl group substituted by an alkyl group having from 1 to 4 carbon atoms, an aralkyl group having 7 or 8 carbon atoms, a halogen atom, an alkoxy group having from 1 to 12 carbon atoms, an aryloxy group, an acyloxy group having from 2 to 18 carbon atoms and its partial ester residue; and two R¹'s and two R⁰'s ay be the same or different.]

(R¹₃Sn)_{d}X′ (iv)

[In the formula, R¹ represents an atom or a group selected from the group consisting of an alkyl group having from 1 to 12 carbon atoms and optionally having a substituent, an alkenyl group having from 2 to 12 carbon atoms, a cycloalkyl group having from 3 to 8 carbon atoms, an aryl group, an aryl group substituted by an alkyl group having from 1 to 4 carbon atoms, an aralkyl group having 7 or 8 carbon atoms, a halogen atom, an alkoxy group having from 1 to 12 carbon atoms, an aryloxy group, an acyloxy group having from 2 to 18 carbon atoms and its partial ester residue; and at least one R¹ is a group selected from the group consisting of an alkyl group having from 1 to 12 carbon atoms and optionally having a substituent, an alkenyl group having from 2 to 12 carbon atoms, a cycloalkyl group having from 3 to 8 carbon atoms, an aryl group, an aryl group substituted by an alkyl group having from 1 to 4 carbon atoms and an aralkyl group having 7 or 8 carbon atoms; X′ represents a group selected from the group consisting of a carbonic acid group, a phosphorus oxyacid group, a partial ester residue of phosphoric acid, a polybasic carboxylic acid group, and a polyalcohol residue; d is an integer larger than 1, corresponding to the basicity of X′.]

A complex comprising a compound of the general formula (i) and a compound of the general formula (ii) may be used as the organotin compound.

Concretely, the compound of the general formula (i) includes the following:

(C₂H₅)₄Sn, (C₆H₅)₄Sn, (CH₃)₃SnF,

(C₄H₉)₃SnCl, (CH₃)₃SnBr, (C₈H₁₇)₃SnCl,

(CH₃)₂SnF₂, (C₄H₉)₂SnCl₂,

(C₁₂H₂₃)₂SnBr₂, (cyclo-C₆H₁₁)₂SnI₂,

(C₄H₉)SnF₃, (C₈H₁₇)SnCl₃,

(C₄H₉)₃SnOC₄H₉, (C₈H₁₇)₃SnOCOCH₃,

(C₈H₁₇)₂(Sn(OCOC₁₇H₃₅)₂

The compound of the general formula (ii) includes the following:

(CH₃)₂SnO, (C₄H₉)₂SnO, (C₈H₁₇)₂SnO,

(C₆H₅)₂SnO, CH₃SnO_{3/2}, C₄H₉SnO_{3/2}

Examples of the complex comprising a compound of the general formula (i) and a compound of the general formula (ii) are the following:

(CH₃)₂SnO·(C₂H₅)₂SnBr₂,

(CH₃)₂SnO·(CH₃)₂SnCl₂,

CH₃{(CH₃)₂SnO}₂CH₃·(CH₃)₂SnBr₂

The compound of the general formula (iii) includes the following:

(CH₃)₃SnOSn(CH₃)₃,

Cl(C₄H₉)₂SnOSn(C₄H₉)Cl,

(CH₃COO) (C₆H₅)SnOSn(C₆H₅) (CH₃COO)

The compound of the general formula (iv) includes the following:

{(CH₃)₃Sn}₂CO₃,

{(C₄H₉)₃Sn}₂CO₃,

(C₄H₉)₃SnOP(O) (OC₈H₁₇)₂,

{(C₈H₁₇)₃Sn}₃PO₄,

(C₄H₉)₃SnOCH₂CH₂OSn(C₄H₉)₃,

(C₄H₉)₂(CH₃O)Sn—OCO—(CH₂)₄—OCO—Sn(OCH₃) (C₄H₉)₂

As the alkyl phosphate, usable is a complete or partial ester of orthophosphoric acid of the following general formula (v):

(R²O)₃P═O (v)

[In the formula, R² represents a hydrogen atom, an alkyl group having from 2 to 12 carbon atoms, an alkenyl group having from or 3 carbon atoms, or a cycloalkyl group having from 3 to carbon atoms; and at least one R² is a group except hydrogen atom.]

Concrete examples of the compound of the general formula (v) are the following:

(C₂H₅)₃PO₄, (C₃H₇)₃PO₄,

(C₄H₉)₃PO₄, (C₈H₁₇)₃PO₄,

(CH₂═CH—CH₂)₃PO₄,

(C₆H₁₁)₃PO₄,

(ClCH₂—CH₂)₃PO₄,

(Cl₂C₃H₅)PO₄, (C₂H₅)₂HPO₄,

(C₄H₉)₂HPO₄, (C₄H₉)H₂PO₄

The catalyst to be used in the production process of the invention comprises a condensate to be obtained by heating a mixture of the above-mentioned organotin compound and alkyl phosphate at a temperature falling within a range of from 150° C. to 300° C. A solvent may be optionally used in the condensation reaction. The organotin compound and the alkyl phosphate are used generally in such a manner that the ratio of the tin atom to the phosphorus atom contained may fall within a range of from 1/10 to 10/1.

In the above condensation reaction, various relatively simple substances are formed and released, depending on the type of the organotin compound and the alkyl phosphate. The obtained condensate shows the intended activity in various stages of the degree of condensation. The optimum degree of condensation may differ depending on the type and the ratio of the organotin compound and the alkyl phosphate, and may be determined experimentally with ease. In general, in the initial stage, the condensate is soluble in a solvent such as hexene, benzene, but becomes insoluble with the reaction going on.

In a more concrete example of catalyst production reaction, dibutyltin oxide as an organotin compound and tributyl phosphate as an alkyl phosphate are put into a reactor, and heated at a temperature falling within a range of from 150° C. to 300° C. with stirring in a nitrogen atmosphere for 1 minutes to 3 hours or so, and while removing the distillate through distillation, a solid condensate is obtained as a residue.

Not specifically defined, the amount of the catalyst to be used is, in general, preferably from 0.01 to 1% by weight relative to the total amount of the monomer and the polymerization solvent, more preferably from 0.05 to 0.5% by weight.

The chain transfer agent to be used in the invention may be any one comprising, as the major ingredient thereof, an aliphatic polyalcohol of the formula (I), preferably comprising, as the major ingredient thereof, an aliphatic dialcohol. More preferred is a chain transfer agent comprising analiphatic polyalcohol, for example, analiphatic dialcohol alone.

Examples of the aliphatic dialcohol are ethylene glycol, 1,2-propanediol, 1,3-propanediol, 1,2-butanediol, 1,4-butanediol, 2,4-pentanediol, 1,5-pentanediol, 1,2-hexanediol, 1,6-hexanediol, 1,7-heptanediol, 1,8-octanediol, 2,5-dimethyl-2,5-hexanediol, 1,4-cyclohexanediol.

Of the aliphatic dialcohol, preferred is an aliphatic dialcohol having 4 or 5 carbon atoms. Of the aliphatic dialcohol having 4 or 5 carbon atoms, preferred is a compound having one alcohol group bonding to both terminals of the carbon chain thereof. Most preferred is 1,4-butanediol.

The amount of the chain transfer agent to be added in the invention may be suitably selected depending on the monomer ratio, the Mooney viscosity and the like of the intended polymer. In general, the amount of the chain transfer agent to be used falls within a range of from 10 ppm to 10000 ppm (by weight) relative to the total weight of the polymerizable monomer and the polymerization solvent. Depending on the combination of the monomer type and the solvent type, additives such as dispersant, stabilizer and the like may be added, if desired, in order that the chain transfer agent may be effectively reactive.

In the process of producing an ether (co) polymer of the invention, the above monomer may be polymerized (preferably in a mode of slurry polymerization), in the presence of the above-mentioned polymerization catalyst and chain transfer agent in a solvent, using a suitable reactor. The solvent used in the reaction is recovered from the reaction mixture after the polymerization reaction, and purified. The recovered solvent does not substantially contain an active hydrogen-containing compound (water, aliphatic dialcohol, etc.), and this can be reused as a solvent in new polymerization reaction.

Not specifically defined, the polymerization reaction temperature maybe generally within a range of from −30 to 150° C. In general, the reaction pressure may well be ordinary pressure. For example, copolymerization of epichlorohydrin and ethylene oxide may be attained under normal pressure at a temperature falling within a range of from 10 to 70° C. Not also specifically defined, the reaction time may be a period of time until the completion of polymerization, generally falling within a range of from 1 to 72 hours.

In slurry polymerization, just after all the components to be fed are fed into a reactor, they are all miscible and may keep a transparent uniform system as a whole. With the reaction going on, the polymerization of the starting monomer goes on and a polymer is gradually deposited, whereby the liquid becomes gradually cloudy and the polymerization is thus completed.

If desired, it is also desirable to use a multi-stage reactor and to make the polymerization conversion in the first stage at most 10% for reducing the polymer adhesion to the wall surface of the polymerization tank, for example, as described in JP-B 61-58488.

Carrying out the production process of the invention gives the intended product, ether (co)polymer. Of the ether (co)polymer, the ether homopolymer is a polymer obtained through polymerization of one monomer selected from a halogen-containing ether monomer (1) and a halogen-free ether monomer (2). The ether copolymer is an ether copolymer obtained through copolymerization of two or more monomers selected from a halogen-containing ether monomer (1) and a halogen-free ether monomer (2), or a polynary copolymer obtained through copolymerization of two or more halogen-free ether monomers (2) with a copolymerizable crosslink site monomer (3).

The ether (co)polymer is preferably an epichlorohydrin homopolymer, a binary copolymer of epichlorohydrin and ethylene oxide, or a ternary copolymer of epichlorohydrin, ethylene oxide and allyl glycidyl ether, as having excellent heat resistance and oil resistance and capable of being widely used in automobile parts, etc.

Not specifically defined, the Mooney viscosity of the ether (co)polymer of the invention is preferably at most 100, more preferably at most 70.

In slurry polymerization, the reaction mixture is separated into the intended product, ether (co)polymer and a liquid component essentially comprising a solvent, after completion of the polymerization reaction, according to a liquid-solid separation method of filtration or the like. The separated liquid component is then purified, and the recovered solvent may be repeated used as a reaction solvent. The used chain transfer agent is removed from the solvent in every solvent purification, and therefore the chain transfer agent is prevented from accumulating in the solvent used repeatedly.

In the invention, a water extraction method is employed for purification of the liquid component essentially comprising a solvent. The water extraction method is, for example, a) a method comprising contacting water with the liquid component to transfer the chain transfer agent dissolving in the solvent into water, then recovering the solvent through evaporation, and distilling the recovered solvent to remove water remaining in the solvent, to thereby remove the chain transfer agent from the solvent; or b) a method comprising adding water to the liquid component, introducing a steam flow into the mixture to recover the solvent through evaporation, then distilling the recovered solvent to remove water remaining in the solvent, to thereby remove the chain transfer agent from the solvent. In any method, the chain transfer agent of the invention may be easily removed from the solvent.

### EXAMPLES

The invention is described in detail with reference to the following Examples. However, the invention should not be limited to these Examples.

### Reference Example 1

### Synthesis of Polymerization Catalyst

10.0 g of dibutyltin oxide and 23.4 g of tributyl phosphate were put into a three-neck flask equipped with a thermometer and a stirrer, and the feedstocks were heated under a nitrogen stream at 260° C. with stirring for 15 minutes to remove the distillate, thereby obtaining a solid condensate as a residue. The condensate was used as a catalyst in the following polymerization reaction.

### Examples 1 to 3

A SUS reactor having a capacity of 20 L and equipped with a thermometer and a stirrer was purged with nitrogen gas, and 5 g of a catalyst of the above condensate, 5 kg of normal hexane having a water content of at most 10 ppm, 0.8 kg of epichlorohydrin (hereinafter abbreviated as EP), and ⅓ of 1.2 kg of ethylene oxide (hereinafter abbreviated as EO) were fed into it; and, as a chain transfer agent, 1,4-butanediol or 2,5-dimethyl-2,5-hexanediol was added in the amount indicated in Table 1, and these were subjected to polymerization reaction at 25° C. for 8 hours. On 2 hours and 4 hours of the reaction time, ⅓ of the remaining amount of EO was added, respectively. The reaction mixture was filtered for solid-liquid separation. The separated solid component was dried under reduced pressure at 70° C. for 24 hours. Thus obtained, the weight of the rubber-like polymer was divided by the monomer weight (2 kg) to obtain the yield.

100 g of the rubber-like polymer was kneaded with a 6-inch roll conditioned at 70° C. and formed into a sheet, and its Mooney viscosity (L rotor) was measured at 100° C. according to the method described in JIS K 6300-1.

The results are shown in Table 1.

### Comparative Examples 1 to 3

The same process as in Example 1 was repeated, except that the chain transfer agent was not used or the chain transfer agent was changed to one shown in Table 1.

These results are shown in Table 1.

TABLE 1

| | | | | | Comparative | Comparative | Comparative |
|---|---|---|---|---|---|---|---|
| | | Example 1 | Example 2 | Example 3 | Example 1 | Example 2 | Example 3 |
| Chain | 1,4-butanediol | 100 | 150 | — | — | — | — |
| Transfer | 2,5-dimethyl-2,5-hexanediol | — | — | 200 | — | — | — |
| Agent | water | — | — | — | — | 250 | — |
| (ppm) | t-butyl alcohol | — | — | — | — | — | 125 |

| | | | | | | |
|---|---|---|---|---|---|---|
| Polymerization Yield (%) | 100 | 100 | 100 | 100 | 1 | 97 |
| Mooney Viscosity of | 60 | 48 | 46 | 103 | not polymerized | 51 |
| Polymer (ML₁₊₄) | | | | | | |

The chlorine content of the rubber-like polymer obtained in the above Examples and Comparative Examples was measured, and the molar % of the EP component and the EO component was computed, and the polymer composition was thus determined. In all Examples and Comparative Examples except Comparative Example 2, the EP component was from 24 to 26 mol % and the EO component was from 76 to 74 mol %, and they well agreed.

### Reference Example 2

500 ml of the liquid component separated in Example 2 and 500 ml of water were put into a 2-L three-neck flask, and the mixture was heated in a water bath whereby normal hexane was completely evaporated; and the evaporated normal hexane was recovered and the amount of the chain transfer agent contained therein was quantitatively determined by gas chromatography. Using 500 ml of the liquid component separated in Comparative Example 3 in place of the liquid component separated in Example 2, the above process was repeated. The results obtained are shown in Table 2.

TABLE 2

| | | Hexane in |
|---|---|---|
| | Hexane in | Comparative |
| | Example 2 | Example 3 |
| Amount of Chain | 80 | 95 |
| Transfer Agent in | | |
| Hexane after | | |
| Separation (ppm) | | |
| Amount of Chain | | |
| Transfer Agent in | 1 or less | 24 |
| Hexane after | | |
| Evaporation (ppm) | | |

### INDUSTRIAL APPLICABILITY

The production process for ether (co)polymer of the invention and the chain transfer agent to be used in it may be effectively utilized in the production field of polyether rubber, especially epichlorohydrin rubber. The ether (co) polymer obtained according to the invention is worth using as vulcanized rubber in charge rolls and development rolls for copiers, printers, etc.

1. A process for producing an ether (co)polymer, wherein an ether monomer is polymerized in a solvent, in the presence of a catalyst comprising a condensate of an organotin compound and an alkyl phosphate, and a chain transfer agent comprising, as the major ingredient thereof, an aliphatic polyalcohol of the following general formula (I), thereby producing an ether (co)polymer:
Cₓ(OH)_{y}H_{z} (I)
wherein X indicates an integer of from 2 to 8; Y indicates an integer of from 2 to (2X +2); Z indicates an integer of 2X+2−Y; and the hydroxyl groups are bonded at any positions.

2. A process for producing an ether (co)polymer, wherein an ether monomer is polymerized in a solvent immiscible with water, in the presence of a catalyst comprising a condensate of an organotin compound and an alkyl phosphate, and a chain transfer agent comprising, as the major ingredient thereof, an aliphatic polyalcohol of the following general formula (I), then the obtained reaction mixture is separated into an ether (co)polymer and a liquid component, and thereafter the liquid component is purified according to a water extraction method, and the solvent is recovered:
Cₓ(OH)_{y}H_{z} (I)
wherein X indicates an integer of from 2 to 8; Y indicates an integer of from 2 to (2X+2); Z indicates an integer of 2X+2−Y; and the hydroxyl groups are bonded at any positions.

3. The process for producing an ether (co)polymer as claimed in claim 1 or 2, wherein the ether monomer polymerization is slurry polymerization.

4. A chain transfer agent for use in the ether (co) polymer production process as claimed in any one of claims 1 to 3, which comprises, as the major ingredient thereof, an aliphatic polyalcohol of the following general formula (I):
Cₓ(OH)_{y}H_{z} (I)
wherein X indicates an integer of from 2 to 8; Y indicates an integer of from 2 to (2X+2); Z indicates an integer of 2X+2−Y; and the hydroxyl groups are bonded at any positions.

5. An ether (co) polymer produced according to the ether (co) polymer production process as claimed in any one of claims 1 to 3.
